(19) United States
(12) Patent Application Publication
(10) Pub. No.: US 2026/0014221 A1
(43) Pub. Date: Jan. 15, 2026

(54) ACID REFLUX AND HEARTBURN CURING COMPOSITION

(71) Applicant: Darlene Moore, Kalamazoo, MI (US)

(72) Inventor: Darlene Moore, Kalamazoo, MI (US)

(21) Appl. No.: 18/769,850

(22) Filed: Jul. 11, 2024

(51) Int. Cl. A61K 36/31; A61K 9/48; A61K 35/741; A61K 36/9066; A61K 36/9068; A61K 38/43

(52) U.S. Cl. CPC A61K 36/31; A61K 9/4816; A61K 35/741; A61K 36/9066; A61K 36/9068; A61K 38/43

(57) ABSTRACT

An easy-to-administer capsule containing a composition for quickly providing relief from acid reflux and heartburn. The capsule is orally administered and the composition includes mustard powder and turmeric powder. In one embodiment, ginger root extract, probiotic blends, and digestive enzyme blends are also included in the composition. The composition starts providing relief within 10 minutes and provides relief for about 12 hours. The composition is available in the form of capsules or powder. The composition is natural and organic and the capsule disintegrates inside the gut of a human.

### FIELD OF THE INVENTION

The present invention generally relates to the field of treating acid reflux and heartburn. More specifically, the present invention relates to a novel composition that is organic, and natural and comes in a capsule or powder form for providing relief from acid reflux and heartburn within 10 minutes and for maintaining relief for about 12 hours after ingesting. The capsule shell storing the composition includes mustard and the capsule disintegrates in the gut of a human for quick absorption of the composition. Accordingly, the present disclosure makes specific reference thereto. Nonetheless, it is to be appreciated that aspects of the present invention are also equally applicable to other like applications, devices, and methods of manufacture.

### BACKGROUND

By way of background, acid reflux is a medical condition in which stomach acid flows backward into the esophagus. The backward flow of acid can lead to different symptoms, including a burning sensation in the chest, which is often referred to as heartburn. Some individuals may experience more severe and chronic forms of acid reflux known as gastroesophageal reflux disease (GERD). GERD can cause frequent and persistent symptoms, potentially leading to complications if left untreated.

Commonly, acid reflux is caused when large meals are consumed or due to eating late at night. When an individual eats a large meal or lies down shortly after eating, pressure is exerted on the lower esophageal sphincter (LES), which is a ring of muscle that prevents stomach acid from flowing back into the esophagus. Certain foods and beverages are also known to relax the LES or increase stomach acid production, making them more likely to trigger acid reflux. Such food and beverage items include fatty or fried foods, spicy foods, citrus fruits, tomato products, chocolate, coffee, and alcohol. High levels of stress and anxiety can also exacerbate acid reflux symptoms in some individuals.

For relief from acid reflux and heartburn, people commonly take medications such as antacids, H2 blockers, and proton pump inhibitors (PPIs) to help reduce stomach acid production and alleviate symptoms. Many medications include chemicals and may have adverse effects on the body. Further, such medications provide relief from heartburn for a very short period of time such as 2 or 3 hours. Many individuals attempt lifestyle changes to relieve heartburn by avoiding trigger foods, eating smaller portions, more frequent meals, not lying down immediately after eating, and losing weight if necessary. However, lifestyle changes are shown to slow the effects. People are looking for quicker, more convenient, and safer methods of curing heartburn while continuing to enjoy eating food items and beverages.

Therefore, there exists a long-felt need in the art for an acid reflux and heartburn medication that is natural and organic. Additionally, there is a long-felt need in the art for an acid reflux and heartburn medication that can be orally administered for an instant reduction in stomach acid buildup. Moreover, there is a long-felt need in the art for an orally administered pill to help alleviate heartburn and acid reflux in a short period of time. Further, there is a long-felt need in the art for an acid reflux and heartburn medication pill that does not contain chemicals and is safe for people of all ages. Furthermore, there is a long-felt need in the art for an oral medication for reducing acid build-up in the stomach even after eating large meals and eating late at night. Finally, there is a long-felt need in the art for an acid reflux and heartburn medication/composition capsule or pill that provides instant relief from acid reflux and heartburn while also providing relief for a considerable duration such as 12 hours, thereby obviating frequent doses of chemical-based medicines.

The subject matter disclosed and claimed herein, in one embodiment thereof, comprises an acid reflux and heartburn capsule. The capsule is orally administered and stores a composition for curing acid reflux and heartburn. The capsule includes a shell made of a binder material such as gelatin or Hypromellose and mustard, the shell stores a composition which includes mustard powder and turmeric powder. The capsule disintegrates inside the gut of a human for quick absorption of both the mustard powder and the turmeric powder. The capsule is effective in curing acid reflux and heartburn within ten (10) minutes and continues curing for a period of up to twelve (12) hours.

In this manner, the acid reflux and heartburn medication/composition capsule and the composition of the present invention accomplish all of the foregoing objectives and provide users with an improved orally administered pill for curing acid reflux and heartburn instantly. The composition is natural, and organic, and therefore, prevents users from having to use over-the-counter pharmacy products containing harmful chemicals. The healing time starts within 10 minutes and provides protection for up to 12 hours.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an acid reflux and heartburn capsule storing a composition for curing acid reflux and heartburn. The capsule includes a shell made of a binder material and mustard, the shell storing a composition including one teaspoon mustard powder and one tablespoon turmeric powder, wherein the capsule is orally administered and disintegrates inside the gut of a human for quick absorption. The capsule is effective in curing acid reflux and heartburn within ten minutes and for a period of up to twelve hours.

In a further embodiment, an orally administered acid reflux and heartburn curing composition is disclosed. The composition includes natural and organic ingredients for healing acid reflux and heartburn within 10 minutes and comprises (i) 10-25 weight percentage mustard powder, (ii) 60-75 weight percentage turmeric powder standardized to contain at least 95% curcuminoids, (iii) 9-12 weight percentage ginger root extract, (iv) probiotic blend, (v) 4-14 digestive enzyme blend, and (vi) 2-8 natural sweetener.

In yet another embodiment, a capsule or pill for oral administration to cure acid reflux and heartburn is disclosed. The capsule includes 23-26% w/w mustard powder and 74-77% w/w turmeric powder, the capsule has the ability to disintegrate inside the human body for curing acid reflux and heartburn within 10 minutes and for up to 12 hours.

In yet another embodiment, the capsules are stored in containers wherein the containers have a volume from about 1 oz. to about 4 oz.

In yet another embodiment, the medication/composition reduces inflammation in the esophagus and stomach lining.

In another aspect of the present invention, the composition reduces pain and the production of acid reflux for 12 hours.

In yet another embodiment, a method for curing acid reflux and heartburn is described. The method includes the steps of oral administration of a capsule, the capsule includes organic and natural ingredients consisting of mustard powder, turmeric powder, ginger root extract, probiotic blend, digestive enzyme blend, and natural sweetener, wherein the method results in reduced inflammation, pain, and production of acid reflux for a period of up to 12 hours.

Numerous benefits and advantages of this invention will become apparent to those skilled in the art to which it pertains upon reading and understanding of the following detailed specifications.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of an acid reflux and heartburn medication capsule of the present invention in accordance with one embodiment;

FIG. 2 illustrates a perspective view of one potential embodiment of a small container storing the acid reflux and heartburn medication/composition capsule of the present invention in accordance with the disclosed architecture; and

FIG. 3 illustrates a flow chart depicting a process of making the acid reflux and heartburn medication capsule of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an acid reflux and heartburn medication that is natural and organic. Additionally, there is a long-felt need in the art for an acid reflux and heartburn medication that can be orally administered for an instant reduction in stomach acid buildup. Moreover, there is a long-felt need in the art for an orally administered pill to help alleviate heartburn and acid reflux in a short time. Further, there is a long-felt need in the art for an acid reflux and heartburn medication pill that does not contain chemicals and is safe for people of all ages. Furthermore, there is a long-felt need in the art for an oral medication for reducing acid build-up in the stomach even after eating large meals and eating late at night. Finally, there is a long-felt need in the art for an acid reflux and heartburn medication/composition capsule or pill that provides instant relief from acid reflux and heartburn while also providing relief for a considerable duration such as 12 hours obviating frequent doses of chemical-based medicines.

The present invention, in one exemplary embodiment, is a method for curing acid reflux and heartburn. The method includes the steps of oral administration of a capsule, the capsule includes organic and natural ingredients consisting of mustard powder, turmeric powder, ginger root extract, probiotic blend, digestive enzyme blend, and natural sweetener, wherein the method results in reduced inflammation, pain, and production of acid reflux for a period of up to 12 hours.

In accordance with the present invention, the natural acid reflux and heartburn medication/composition for curing and preventing acid reflux comprises only natural and organic ingredients. The medication/composition can be provided in the form of a capsule or the form of a mix of capsule and powder. The composition consists of mustard powder and turmeric powder. In some embodiments, the composition includes mustard powder, turmeric powder, ginger root extract, probiotic blend, and digestive enzyme blends. The medication can be used by a person for oral administration without requiring a prescription from a medical doctor.

Mustard powder helps with acid reflux and heartburn due to the alkaline properties of the mustard. The alkaline properties of the mustard powder help neutralize stomach acid. Consuming the mustard powder reduces the flow back of stomach acid into the esophagus. Mustard also creates a protective coating along the lining of the esophagus and stomach and helps prevent further irritation and provides a soothing effect, reducing the discomfort associated with acid reflux. The strong flavor of the mustard powder stimulates saliva production. Saliva is a natural antacid, and increased saliva production helps neutralize stomach acid and promote digestion. The amount of mustard powder present in each dose of the compositions of the acid reflux and heartburn medication/composition ranges from about 10 weight percent to about 25 weight percent.

Turmeric includes an active compound called curcumin, which has anti-inflammatory properties and reduces inflammation in the esophagus and stomach lining. Curcumin also has gastroprotective effects which may help protect the stomach lining from damage caused by excessive stomach acid, which can contribute to heartburn and acid reflux. Turmeric also stimulates bile production, which aids in digestion. Improved digestive process due to turmeric can reduce the likelihood of food and stomach acid regurgitating into the esophagus. The turmeric used in the medication is standardized to contain at least 95% curcuminoids. The amount of turmeric present in each dose of the compositions of the acid reflux and heartburn medication/composition ranges from about 60 weight percent to about 75 weight percent.

Ginger root extract has anti-inflammatory properties which helps to reduce inflammation in the esophagus. Antioxidant properties of ginger root extract protect cells from damage. Ginger root extract also helps to increase mucus production in the stomach, which can help to protect the stomach lining from stomach acid. The amount of ginger root extract present in each dose of the compositions of the acid reflux and heartburn medication/composition ranges from about 9 weight percent to about 12 weight percent.

Probiotic blends preferably including Lactobacillus and Bifidobacterium strains are used which promote gut health by restoring the balance of beneficial bacteria. The probiotic blend also helps in reducing inflammation and improving digestion and can be in the range from about 10 billion colony-forming units (CFU) to about 20 billion CFU in the medication/composition.

Digestive enzyme blend including amylase, protease, lipase, and cellulase, is included in the composition/medication to aid in the breakdown of various nutrients and to help relieve from heartburn and indigestion.

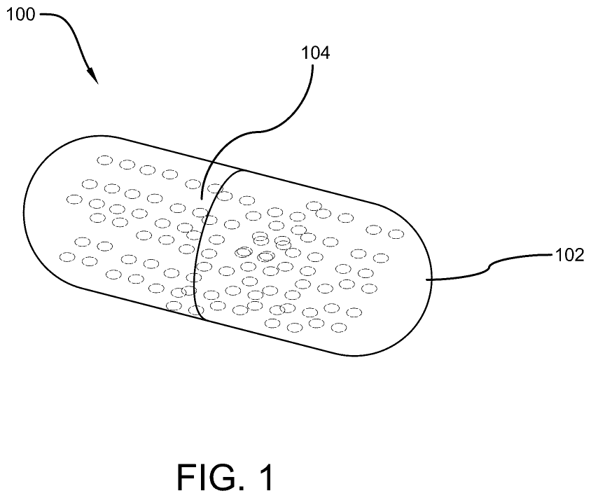

Referring initially to the drawings, FIG. 1 illustrates a perspective view of an acid reflux and heartburn medication capsule of the present invention in accordance with one embodiment. The acid reflux and heartburn medication/composition capsule 100 is used for curing acid reflux and heartburn within ten minutes and for preventing discomfort for about twelve hours after ingestion. Further, the capsule or pill 100 can be orally administered with water without the prescription of any doctor or medical professional. The acid reflux and heartburn medication/composition capsule 100 has a shell 102 for providing protection and containment for the medication/composition 104. The shell 102 is made of mustard and a binder such as gelatin or Hypromellose. The medication is filled inside the shell 102 and is natural and organic, preferably in a powder form. The capsule 100 is designed to disintegrate or dissolve quickly in the digestive tract to release the medication 104 for absorption for quick relief from acid reflux and heartburn.

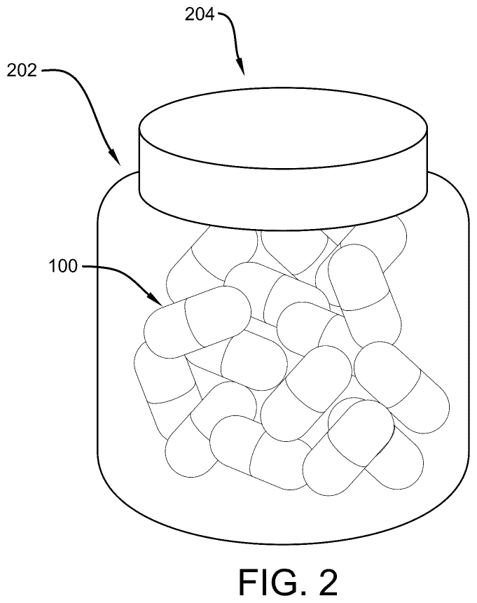

FIG. 2 illustrates a perspective view of one potential embodiment of a small container storing the acid reflux and heartburn medication/composition capsule of the present invention in accordance with the disclosed architecture. The acid reflux and heartburn medication/composition capsule 100 of the present invention is orally administered for curing acid reflux and heartburn. The capsules 100 are commercially available in small containers 202 available in various sizes, such as 1 oz., 2 oz., 3 oz., 4 oz., and more. The capsule 100 may change color over time due to the natural and organic ingredients. The container 202 may store capsules therein and a logo printed on an exterior surface of the container and also include a removable lid 204 for protecting the stored capsules.

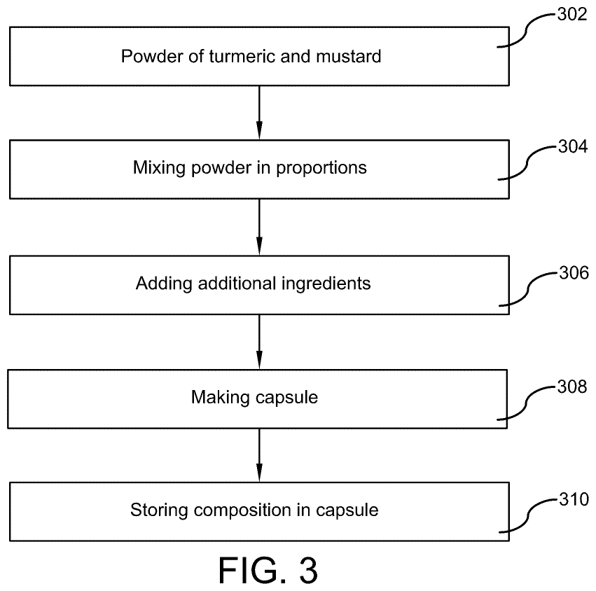

FIG. 3 illustrates a flow chart depicting a process of making the acid reflux and heartburn medication capsule 100 of the present invention in accordance with the disclosed structure. Initially, the turmeric such as turmeric root and mustard seeds are crushed and processed into a fine powder (Step 302). The proportions of the turmeric powder and the mustard powder are then mixed to form the composition (Step 304). Thereafter, depending upon the requirements, additional ingredients such as blends and ginger extract are added into the composition (Step 306). For making the shell, binder materials such as Hydroxypropyl Methylcellulose and mustard are mixed (Step 308). Finally, the composition is stored in individual capsules for subsequent oral administration to cure acid reflux and heartburn (Step 310).

Table 1 below shows approximate concentrations of various ingredients in the acid reflux and heartburn medication/composition 104.

TABLE 1

| | Composition in percentage by weight |
|---|---|
| Ingredients | (% w/w) |
| Mustard powder | 23-26 |
| Turmeric powder | 74-77 |

Table 2 below shows approximate concentrations of various ingredients in another acid reflux and heartburn medication/composition 104.

TABLE 2

| | Composition in percentage by weight |
|---|---|
| Ingredients | (% w/w) |
| Mustard powder | 10-25 |
| Turmeric powder | 60-75 |
| Ginger root extract | 9-12 |
| Probiotic blend | 10 billion-20 billion CFU |
| Digestive enzyme blend | 4-14 |
| Natural Sweetener | 2-8 |

In some embodiments, the composition is in the form of a powder stored in a container. In other embodiments, one dose may include one teaspoon of mustard powder with one tablespoon of turmeric.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “acid reflux and heartburn medication/composition capsule”, “capsule”, “pill”, and “acid reflux buildup prevention capsule” are interchangeable and refer to the acid reflux and heartburn composition capsule 100 of the present invention.

Notwithstanding the foregoing, the acid reflux and heartburn composition capsule 100 of the present invention can be of any suitable size and configuration as is known in the art without affecting the overall concept of the invention, provided that it accomplishes the above-stated objectives. One of ordinary skill in the art will appreciate the acid reflux and heartburn composition capsule 100 as shown in the FIGS. is for illustrative purposes only, and that many other sizes and shapes of the acid reflux and heartburn composition capsule 100 are well within the scope of the present disclosure. Although the dimensions of the acid reflux and heartburn composition capsule 100 are important design parameters for user convenience, the acid reflux and heartburn composition capsule 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An acid reflux and heartburn curing composition comprising:
a capsule for oral administration;
wherein said capsule having a dissolvable shell including a mustard and a binder;
wherein said composition is contained inside said shell; and
further wherein said composition having a mixture of a turmeric root and mustard seeds.

2. The acid reflux and heartburn curing composition of claim 1, wherein said turmeric root is in powder form and said mustard seeds are in powder form.

3. The acid reflux and heartburn curing composition of claim 2, wherein said composition further having a ginger extract.

4. The acid reflux and heartburn curing composition of claim 3, wherein said binder selected from the group consisting of a gelatin, a Hydroxypropyl Methylcellulose, and a Hypromellose.

5. The acid reflux and heartburn curing composition of claim 2, wherein said composition is in a powder form.

6. The acid reflux and heartburn curing composition of claim 2, wherein said composition having a weight percentage of said turmeric powder from 74% to 77% and a weight percentage of said mustard powder from 23% to 26%.

7. An acid reflux and heartburn curing composition comprising:
a capsule for oral administration;
wherein said capsule having a dissolvable shell including a mustard and a binder;
wherein said composition is contained inside said shell; and
further wherein said composition having a mixture of a turmeric powder, a mustard powder, a ginger root, a probiotic blend, a digestive enzyme blend, and a natural sweetener.

8. The acid reflux and heartburn curing composition of claim 7, wherein said turmeric powder having a weight percentage of said composition between 60 and 75 percent.

9. The acid reflux and heartburn curing composition of claim 8, wherein said mustard powder having a weight percentage of said composition between 10 and 25 percent.

10. The acid reflux and heartburn curing composition of claim 9, wherein said ginger root having a weight percentage of said composition between 9 and 12 percent.

11. The acid reflux and heartburn curing composition of claim 10, wherein said probiotic blend including between 10 billion and 20 billion colony-forming units.

12. The acid reflux and heartburn curing composition of claim 11, wherein said digestive enzyme blend having a weight percentage of said composition between 4 and 14 percent.

13. The acid reflux and heartburn curing composition of claim 12, wherein said natural sweetener having a weight percentage of said composition between 2 and 8 percent.

14. The acid reflux and heartburn curing composition of claim 13, wherein said binder selected from the group consisting of a gelatin, a Hydroxypropyl Methylcellulose, and a Hypromellose.

15. The acid reflux and heartburn curing composition of claim 13, wherein said composition is in a powder form.

16. An acid reflux and heartburn curing composition comprising:
a mixture of a turmeric powder, a mustard powder, a ginger root, a probiotic blend, a digestive enzyme blend, and a natural sweetener;
wherein said turmeric powder having a weight percentage of said composition between 60 and 75 percent;
wherein said mustard powder having a weight percentage of said composition between 10 and 25 percent;
wherein said ginger root having a weight percentage of said composition between 9 and 12 percent;
wherein said digestive enzyme blend having a weight percentage of said composition between 4 and 14 percent; and
further wherein said natural sweetener having a weight percentage of said composition between 2 and 8 percent.

17. The acid reflux and heartburn curing composition of claim 16, wherein said probiotic blend including between 10 billion and 20 billion colony-forming units.

18. The acid reflux and heartburn curing composition of claim 17, wherein said composition having a capsule for oral administration;
wherein said capsule having a dissolvable shell;
wherein said composition is contained inside said shell; and
further wherein said dissolvable shell including a mustard and a binder.

19. The acid reflux and heartburn curing composition of claim 18, wherein said binder selected from the group consisting of a gelatin, a Hydroxypropyl Methylcellulose, and a Hypromellose.

20. The acid reflux and heartburn curing composition of claim 16, wherein said composition is in a powder form.
